(19) United States
(12) Patent Application Publication
(10) Pub. No.: US 2010/0212944 A1
(43) Pub. Date: Aug. 26, 2010

(54) Silane Coupling Agents for Printed Circuit Boards

(75) Inventors: Jeffrey Donald Gelorme, Burlington, CT (US); Joseph Kuczynski, Rochester, MN (US)

Correspondence Address: WOOD, HERRON & EVANS, L.L.P. (IBM), 2700 CAREW TOWER, 441 VINE STREET, CINCINNATI, OH, 45202, US

(73) Assignee: INTERNATIONAL BUSINESS MACHINES CORPORATION, Armonk, NY (US)

(21) Appl. No.: 12/694,005

(22) Filed: Jan. 26, 2010

Related U.S. Application Data
(63) Continuation-in-part of application No. 12/391,500, filed on Feb. 24, 2009, now abandoned.

(51) Int. Cl. H05K 1/03; C07D 251/04; H05K 1/02; B05D 5/12

(52) U.S. Cl. 174/258; 544/221; 174/259; 427/97.6

(57) ABSTRACT

The present invention is directed to silane coupling agents for printed circuit boards, such as high-temperature printed circuit boards (PCBs), a process for synthesizing the silane coupling agent, and PCBs with such coupling agents. The silane coupling agent is defined by the following formula:

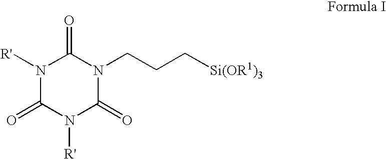

wherein at least one R′ is an allyl and the other one is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl.

### CROSS REFERENCE TO RELATED APPLICATIONS

The is a continuation in part application of and claims priority to application Ser. No. 12/391,500 entitled SILANE COUPLING AGENTS FOR PRINTED CIRCUIT BOARDS.

### FIELD OF THE INVENTION

The present invention is directed to silane coupling agents, and more particularly, to silane coupling agents for printed circuit boards, such as high-temperature printed circuit boards.

### BACKGROUND OF THE INVENTION

The basic concept behind a coupling agent is to join two disparate surfaces. In the case of printed circuit boards (PCBs), a silane coupling agent is often used to join a coating, e.g., a varnish such as an epoxy-based resin, to a substrate, e.g. glass cloth, to define a laminate, or laminated structure. The silane coupling agent typically consists of an organofunctional group to bind to the coating and a hydrolyzable group that binds to the surface of the substrate. In particular, the alkoxy groups on silicon hydrolyze to silanols, either through the addition of water or from residual water on the inorganic surface. Subsequently, the silanols react with hydroxyl groups on the inorganic surface to form an oxane bond (Si—O—Si) and eliminate water.

One problem experienced with PCBs is conductive anodic filament (CAF), which is a copper corrosion byproduct that emanates from the anode of a circuit and “grows” subsurface toward the cathode, frequently along separated fiber-epoxy interfaces. With PCBs, anode/cathode pairs are typically plated through holes. CAF can result in catastrophic failure of PCBs, which in some instances can cause fires. The bond between the varnish and substrate is understood to be an important factor in CAF. Thus, the silane coupling agent is an important consideration in the laminate.

For the specific case of epoxy-based laminates, the organofunctional group that has been found to exhibit desirable performance based on numerous criteria is vinylbenzylaminoethylaminopropyl and also benzylaminoethylaminopropyl. Silane coupling agents, which include this organofunctional group, are thought to covalently bond to the epoxide functional groups of the traditional epoxy-based resin, such as the well known FR4 epoxy resins, through the secondary nitrogens of the amino groups. While a plethora of silane coupling agents exist, the industry workhorse for coupling epoxy-based resins has been vinylbenzylaminoethylaminopropyl-trimethoxysilane (commercially available as Dow Corning Z-6032).

As the PCB industry has migrated away from traditional FR4 epoxy resins (due to lead-free requirements and the higher soldering temperatures associated with tin-silver-copper alloys), silane coupling agents originally developed for those epoxies have not kept pace. That is, vinylbenzylaminoethylaminopropyltrimethoxy silane is still the coupling agent utilized to couple, or bond, the glass cloth substrate to the laminate varnish. However, current varnishes are no longer comprised of FR4 epoxies, rather they are more likely to be bismaleimide triazine (BT) resins or polyphenylene oxide/trially-isocyanurate (PPO/TAIC) interpenetrating networks.

Consequently, a need exists in the industry for a silane coupling agent designed for current resin systems.

### SUMMARY OF THE INVENTION

In accordance with an embodiment of the present invention, a silane coupling agent for printed circuit boards is provided and defined by the following formula:

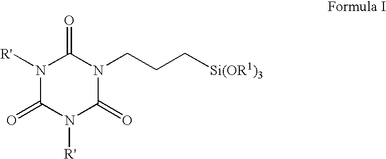

wherein at least one R′ is an allyl and the other is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl. In one example, the silane coupling agent is:

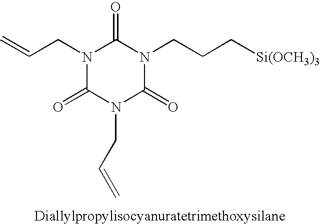

In another embodiment, a process for synthesizing the silane coupling agent is provided. The process includes, in the presence of heat and a catalyst, reacting HSi(OR¹)₃ and

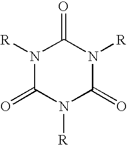

wherein at least two R groups are an allyl and the remaining R group is hydrogen, an alkyl, a cycloalkyl, an aryl, a heteroaryl, a non-aromatic heterocyclic ring, or an allyl and R¹ is as defined above so as to synthesize the silane coupling agent of Formula I. In one example, the resulting silane coupling agent is diallylpropylisocyanurate-trimethoxysilane.

In yet another embodiment, a printed circuit board is provided which includes a substrate, such as a glass cloth, and a varnish coating, such as a polyphenylene oxide/triallylisocyanurate resin, on the substrate. The printed circuit board further includes a silane coupling agent situated between the varnish coating and substrate so as to couple the varnish coating to the substrate. The silane coupling agent is defined by the Formula I, as discussed above.

In still another embodiment, a method of making a printed circuit board is provided which includes applying a silane coupling agent to a substrate, such as a glass cloth. Next, a varnish coating, such as a polyphenylene oxide/triallylisocyanurate resin, is applied on the silane coupling agent so that the silane coupling agent is situated between the varnish coating and substrate. Then, the substrate, coupling agent, and varnish coating are subjected to curing conditions, e.g., heat under vacuum, so as to define a printed circuit board. The silane coupling agent is defined by the Formula I, as discussed above.

### DETAILED DESCRIPTION

In accordance with an embodiment of the invention, there is provided a silane coupling agent for printed circuit boards (PCBs), such as high-temperature PCBs.

The silane coupling agent is defined by the following formula:

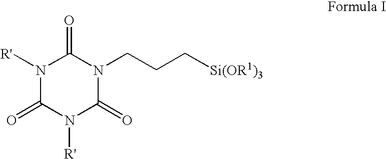

wherein at least one R′ is an allyl and the other one is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl.

In one example, each R′ is an an allyl. In another, example, R¹ is an alkyl group. In yet another example, each R′ is an allyl and R¹ represents an alkyl group having 1 to 18 carbon atoms. In another example, at least one R¹ group is a methyl group. In still another example, the silane coupling agent is:

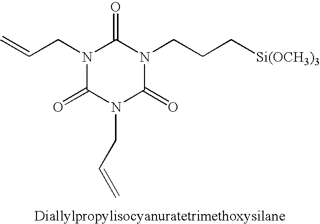

The silane coupling agent of Formula I incorporates an allylisocyanurate ligand as the organofunctional group and is designed to interact with current resin systems, such as polyphenylene oxide (PPO)/triallylisocyanurate (TAIC) resins. In particular, the allyl group(s) of the silane coupling agent can co-react with the allyl group(s) of the TAIC as the varnish cures to form a crosslinked resin tightly bound to the glass fibers of the substrate. Although the polyphenylene oxide (PPO)/triallylisocyanurate (TAIC) resins have been identified herein as suitable for coupling to glass fiber substrates via the silane coupling agent, it is contemplated that any ethylenic unsaturated monomer may be used as a varnish. To that end, in addition to TAIC (or its diallyl analog), vinyl- or methacryl-terminated PPO, any of the diallyl ethers of bisphenol (as described in U.S. Pat. No. 4,774,282, the contents of which is expressly incorporated by reference herein in its entirety), or allyl-modified aryl cyanate esters (e.g., 1-allyl-2-cyanatobenzene), and the like, may be used.

And, while the silane coupling agent generally may be synthesized by methods known in the art, a schematic diagram outlining a proposed reaction mechanism for synthesizing the silane coupling agent of Formula I is shown below.

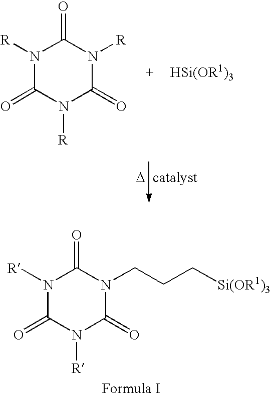

In the above schematic, concerning the allylisocyanurate ligand, at least two of the R groups are an allyl and the remaining R group is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl. In one example, the allylisocyanurate ligand is TAIC, i.e., each R group is an ally, and the silicon hydride compound is trimethoxysilicon hydride, HSi(OCH₃)₃, i.e., R¹ is a methyl, such that the resulting silane coupling agent is diallylpropylisocyanurate trimethoxysilane. In another example, each R is an allyl. In yet another example, R¹ is an alkyl group. In still another example, each R is an allyl and R¹ represents an alkyl group having 1 to 18 carbon atoms.

Further concerning synthesis of the silane coupling agent, the allylisocyanurate ligand is reacted with the silicon hydride compound to reduce the alkene functionality of the allylisocyanurate ligand by adding across a double bond. This reaction takes place in the presence of heat (Δ) and a catalyst, such as transition metal catalyst, e.g., chloroplatinic acid. Judicious selection of the reaction conditions and stoichiometry is thought to limit the reactivity to a single allyl group of TAIC. The catalyst can be used at the 3-20 weight percent range based on total weight of reactants. In another example, the weight percent range can be from 4-8 and, in another example, the weight percent can be 5. The reaction temperature can range from 55-120° C. The completeness of the reaction can be determined by measuring the exotherm (via differential scanning calorimetry). Representative reaction times are in the 15-20 hr range. The reaction can be carried out in toluene or other suitable solvents. The reaction product can be isolated by stripping off the solvent via rotary evaporation or other suitable technique known to those skilled in the art. And, if necessary, fractional distillation can be used to separate the allylpropyl-isocyanurate derivatives.

Concerning the catalyst, transition metal catalysts, such as platinum catalysts, are suitable for use in the synthesis process. Examples of platinum catalysts include chloroplatinic acid as well as those disclosed in U.S. Pat. No. 5,260,399, the contents of which is expressly incorporated by reference herein in its entirety. Also, Wilkinson's catalyst, RhCl[(C₆H₅)₃P]₃], can be used. Ashby's Catalyst (1.75 wt percent platinum in ethanol) can also be used for such hydrosilation reactions, as well as the Lamoreaux catalyst (3.5 wt % platinum in octanol), and the Karstedt catalyst (Pt₂{[(CH₂═CH)Me₂Si])₂O}])₃}]). And, other representative examples include RhX(CO)(PR₃)₂; RhH(CO)[(C₆H₅)₃P]₃; IrCl(CO)[(C₆H₅)₃P]₂; IrI(CO)[(C₆H₅)₃P]₂; IrH(CO)[(C₆H₅)₃P]₃; Pt[(C₆H₅)₃P]₄; trans-Pt[(C₆H₅)₃P]₂Cl₂; cis-Pt[(C₆H₅)₃P]₂C₁₂; Pt[(C₆H₅)₃P]₂(C₂H₄); Pt[(C₆H₅)₃P]₂O₂; trans-PtHCl[(C₆H₅)₃P]₂; cis-PtHCl[(C₆H₅)₃P]₂; Pt(PF₃)₄; Pd[(C₆H₅)₃P]₂Cl₁₂: Pd[(C₆H₅)₃P]₄; Pd[(C₆H₅)₃P]₂(CH₃CO₂)₂; Ni[(C₆H₅)₃P]₄; Ni[(C₆H₅)₃P]₂Cl₂; Co[(C₆H₅)₃P]₂Cl₂; and Ru[(C₆H₅)₃P]₃Cl₂.

The resulting silane coupling agent of Formula I can couple, or bond, the varnish coating to the PCB substrate, e.g. glass cloth, to define a laminate, or laminated PCB. In particular, the resulting silane coupling agent can be applied to the substrate via known means, such as a dip coating process. Then, the treated substrate, e.g., glass substrate, is coated with a varnish, such as PPO/TAIC varnish, via means known in the art. After which, the laminate is subjected to curing conditions, e.g., heated under vacuum, as known in the art, which results in a crosslinked phase that is covalently bound to the glass fibers. And, as with the commercially available silanes, hydrolysis of the trialkoxy groups yields a silanol that reacts with hydroxyl groups on the glass surface to form Si—O—Si bonds upon loss of water. Additionally, the propyl group to which the trialkoxysilane is attached is hydrophobic, further retarding ingress of water to the resin/glass interface and improving CAF resistance.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative product and/or method and examples shown and described. Accordingly, departures may be made from such details without departing from the scope of the general inventive concept.

1. A silane coupling agent defined by the following formula: 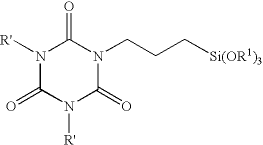
wherein at least one R′ is an allyl and the other is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl.

2. The silane coupling agent of claim 1 wherein each R′ is an allyl.

3. The silane coupling agent of claim 1 wherein R¹ is an alkyl group.

4. The silane coupling agent of claim 1 wherein each R′ is an allyl and R¹ is an alkyl group having 1 to 18 carbon atoms.

5. A silane coupling agent of the following structure:

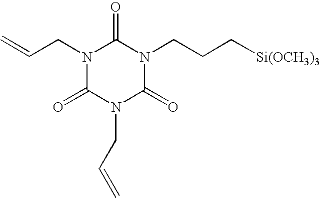

6. A printed circuit board comprising:
a substrate;
a varnish coating on the substrate; and
a silane coupling agent situated between the varnish coating and substrate so as to couple the varnish coating to the substrate, the silane coupling agent defined by the following formula: 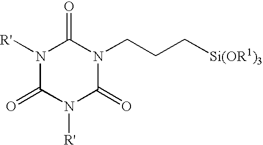
wherein at least one R′ is an allyl and the other one is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl.

7. The printed circuit board of claim 6 wherein each R′ is an allyl.

8. The printed circuit board of claim 6 wherein R¹ represents an alkyl group.

9. The printed circuit board of claim 6 wherein each R′ is an allyl and R¹ represents an alkyl group having 1 to 18 carbon atoms.

10. The printed circuit board of claim 6 wherein the silane coupling agent is

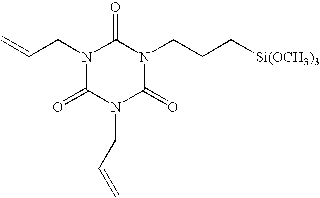

11. The printed circuit board of claim 6 wherein the substrate includes glass and the varnish coating is a polyphenylene oxide/triallylisocyanurate resin.

12. A process for synthesizing a silane coupling agent comprising:
in the presence of heat and a catalyst, reacting 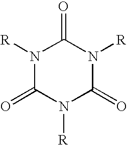
with HSi(OR¹)₃, wherein at least two R groups are an allyl and the remaining R group is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl, so as to synthesize a silane coupling agent defined by the following formula: 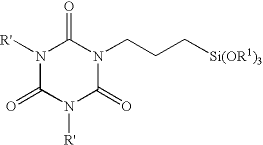
wherein at least one R′ is an allyl and the other one is hydrogen, an alkyl, a cycloalkyl, an aryl, a heteroaryl, a non-aromatic heterocyclic ring, or an allyl; and R¹ is the same as defined above.

13. The process of claim 12 wherein triallylisocyanurate is reacted with HSi(OCH₃)₃ to provide

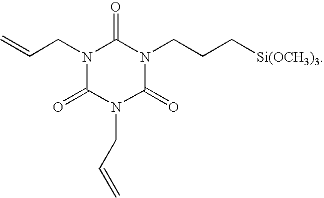

14. The process of claim 12 wherein the heat is from about 55° C. to about 120° C.

15. The process of claim 12 wherein the catalyst is a transition metal catalyst.

16. The process of claim 12 wherein each R is an allyl.

17. The process of claim 12 wherein R¹ is an alkyl group.

18. The process of claim 12 wherein each R is an allyl and R¹ represents an alkyl group having 1 to 18 carbon atoms.

19. A method of making a printed circuit board comprising:
applying a silane coupling agent to a substrate, the silane coupling agent defined by the following formula: 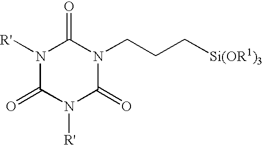
wherein at least one R′ is an allyl and the other one is hydrogen, an alkyl, a cycloalkyl, an aryl, a heteroaryl, a non-aromatic heterocyclic ring, or an allyl; and R¹ is an alkyl, a cycloalkyl, an aryl, a heteroaryl, a non-aromatic heterocyclic ring, or an allyl;
applying a varnish coating on the silane coupling agent wherein the silane coupling agent is situated between the varnish coating; and
subjecting the substrate, silane coupling agent, and varnish coating to curing conditions so as to define a printed circuit board.

20. The method of claim 19 wherein each R′ is an allyl.

21. The method of claim 19 wherein R¹ represents an alkyl group.

22. The method of claim 19 wherein each R′ is an allyl and R¹ represents an alkyl group having 1 to 18 carbon atoms.

23. The method of claim 19 wherein the silane coupling agent is

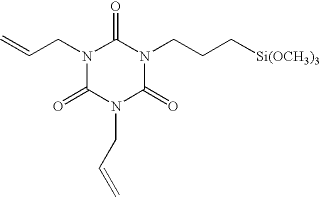

24. The method of claim 19 wherein the substrate includes glass and the varnish coating is a polyphenylene oxide/triallylisocyanurate resin.
